(19) United States
(12) Patent Application Publication
(10) Pub. No.: US 2023/0125212 A1
(43) Pub. Date: Apr. 27, 2023

(54) MEANS AND METHODS FOR ENHANCING CANNABINOIDS EFFICACY

(71) Applicant: Ami MOLAD, Tel Aviv (IL)

(72) Inventor: Ami MOLAD, Tel Aviv (IL)

(21) Appl. No.: 17/915,233

(22) PCT Filed: Mar. 29, 2021
(86) PCT No.: PCT/IL2021/050353

§ 371 Date: Sep. 28, 2022

Related U.S. Application Data
(60) Provisional application No. 63/001,335, filed on Mar. 29, 2020.

(51) Int. Cl. A61K 36/31; A61K 36/82; A61K 36/185; A61K 36/22; A61K 36/48; A61K 36/63; A61K 31/05; A61K 31/01; A61K 31/164; A61K 31/352; A61P 25/08; A61P 1/08; A61P 25/28; A61P 29/00; A61P 11/02; A61K 31/7048; A61P 35/00; A61K 9/00; A61K 36/17; A61P 11/00; A61P 1/14

(52) U.S. Cl. CPC A61K 36/31; A61K 36/82; A61K 36/185; A61K 36/22; A61K 36/48; A61K 36/63; A61K 31/05; A61K 31/01; A61K 31/164; A61K 31/352; A61P 25/08; A61P 1/08; A61P 25/28; A61P 29/00; A61P 11/02; A61K 31/7048; A61P 35/00; A61K 9/007; A61K 36/17; A61P 11/00; A61P 1/14

(57) ABSTRACT

The present invention discloses means and methods for enhancing cannabinoids efficacy. The invention relates to invigorating performance of cannabinoid by means of combining compounds to prime the binding cannabinoid receptors and promoting demethylation processes. Specifically, the present invention discloses a plant-based composition configured to enhance a physiological effect of a cannabis-derived compound, wherein said composition comprises a synergistic combination of the cannabis-derived compound, at least one portion of at least one plant comprising a methylating/demethylating active agent, and at least one portion of at least one enhancer plant.

### FIELD OF THE INVENTION

The invention generally pertains to means and methods for enhancing cannabinoids efficacy.

More specifically, the invention relates to invigorating performance of cannabinoid by means of combining compounds to prime the binding cannabinoid receptors and promoting demethylation processes.

### BACKGROUND OF THE INVENTION

Cannabinoid receptors, the molecular targets of the cannabis-derived compounds, are present throughout the body and are normally bound by a family of endogenous lipids—the endocannabinoids. Release of endocannabinoids is stimulated in a receptor-dependent manner by neurotransmitters and requires the enzymatic cleavage of phospholipid precursors present in the membranes of neurons and other cells. Once released, the endocannabinoids activate cannabinoid receptors on nearby cells and are rapidly inactivated by transport and subsequent enzymatic hydrolysis. These compounds might act near their site of synthesis to serve a variety of regulatory functions, some of which are now beginning to be understood. Recent advances in the biochemistry and pharmacology of the endocannabinoid system are reveling the opportunities that this system offers for the development of novel therapeutic agents.

The most studied and established roles for cannabinoid therapies include pain, chemotherapy-induced nausea and vomiting, and anorexia. Moreover, given their breadth of activity, cannabinoids could be used to concurrently optimize the management of multiple symptoms, thereby reducing overall polypharmacy. The use of cannabinoid therapies could be effective in improving quality of life and possibly modifying malignancy by virtue of direct effects and in improving compliance or adherence with disease-modulating treatments such as chemotherapy and radiation therapy. There is an urgent unmet clinical need for the use of cannabis or cannabis-derived compounds, in a range of disorders that can impair or destroy quality of life and are led by urgent unmet clinical need.

Furthermore, there is additionally an unmet need for enhancing the efficacy of cannabinoids.

### BRIEF DESCRIPTION OF THE FIGURES

FIG. 1 depicts results of the epilepsy study, as Cumulative Seizure Score;

FIG. 2 depicts the average body weight gain in the groups treated with both Can-Epilepsy formulation (FIG. 2A) and Can-LC formulation (FIG. 2B), at three doses

### SUMMARY OF THE INVENTION

It is one object of the invention to disclose a plant-based composition configured to enhance a physiological effect of a cannabis-derived compound, wherein said composition comprises a synergic combination of said cannabis-derived compound, at least one portion of at least one plant comprising a methylating/demethylating active agent, and at least one portion of at least one enhancer plant.

It is one object of the invention to disclose a plant-based composition configured to enhance a physiological effect of a cannabis-derived compound in the treatment of seizures in epilepsy, wherein said composition comprises a synergistic combination of said cannabis-derived compound, at least one portion of at least one plant comprising a methylating/demethylating active agent, and at least one portion of at least one enhancer plant,

wherein said cannabis-derived compound is CBD;

said at least one portion of at least one plant comprising a methylating/demethylating active agent is Hibiscus tiliaceus Linn comprising Dimethylglycine; and

said at least one portion of at least one enhancer plant, is selected from a group consisting of mango, myrcene, palmitoylethanolamide, fenugreek and any combination thereof.

It is one object of the invention to disclose a plant-based composition configured to enhance a physiological effect of a cannabis-derived compound in the treatment of lung cancer, wherein said composition comprises a synergistic combination of said cannabis-derived compound, at least one portion of at least one plant comprising a methylating/demethylating active agent, and at least one portion of at least one enhancer plant,

wherein said cannabis-derived compound is CBD;

said at least one portion of at least one plant comprising a methylating/demethylating active agent is olive, comprising Hydroxytyrosol; and

said at least one portion of at least one enhancer plant, is selected from a group consisting of mango, myrcene, palmitoylethanolamide, fenugreek and any combination thereof.

It is another object of the invention to disclose a composition, as defined in any of the above wherein said physiological effect is selected from a group consisting of increased bioavailability, increased appetite, amelioration of a disease condition, and any combination thereof, said disease condition is selected from a group consisting of nausea post chemotherapy, vomiting post chemotherapy, multiple sclerosis, nerve injury, ocular pain, headache, ADHD, anxiety, insomnia, convulsions, multiple sclerosis, cerebral ischemia, Parkinson's disease, epilepsy, osteoarthritis, psoriasis, systemic lupus erythematosus, diabetes, glomerulonephritis, renal ischemia, nephritis, hepatitis, vasculitis, myocardial infarction, inflammatory bowel disease (IBD), colitis, emesis, Crohn's disease, immune-related disorders, pain-related disorders, and any combination thereof.

It is another object of the invention to disclose a composition, as defined in any of the above, wherein said composition is further configured to target said physiological effect specific body parts or physiological systems.

It is another object of the invention to disclose a composition, as defined in any of the above, wherein said enhancer plant is selected from a group consisting of Maticaria chamimilla, Subgenus Pinus, Lavandula officinalis, Piper nigrum, Mangifera, Fenugreek, Citrus limon and any combination thereof.

It is another object of the invention to disclose a composition, as defined in any of the above, wherein said demethylating-methylating plant is selected from a group consisting of: Medicago sativa, Aloe succotrina, Anabasis alexandri, Hordeum Spontaneum, Hordeum spontaneum, Pimpinella Anisum, Mercurialis annua, Malus sylvestris, Cynara Scolymus, Mandragora autumnalis, Cynodon Dactylon, Citrullkus colocynthis, Nigella Sativa, Solaqnum nigrum, Eucalyptus camaldulensis, Theobroma Cacao, Quercus calliprinos, Oxalis pes-caprae, Carum carvi, Elettaria cardamomum, Daucus Carota, Sinapis Arvensis, Prunus (cerasus) avium, Allium schoenoprasum, Ziziphus spina-christi, Citrus medica, Kaolin, Eugenia caryophyllata, Caffea Arabica, Foeniculum vulgare, Glycyrrhiza glabra, Myrtus communis, Ruta chalepensis, Salvia officinalis, Valeriana officinalis, Coriandrum sativum, Papaver umbonatum, Zea mays, Chrysanthemum coronarium, Cucumis sativus, Cuminum cyminum, Phoenix dactylifera, Origanum dayi, Cichorium pumilum Rubia tenuifolia, Echinacea purpurea, Sambucus nigra, Lycium europaeum, Oenothera drummondii, Digitalis purpurea, Lvandula stoechas, Hibiscus, sea hibiscus, beach hibiscus, coastal (or coast) hibiscus, coastal (or coast) cottonwood, green cottonwood, native hibiscus, native rosella, cottonwood hibiscus Hibiscus tiliaceus Linn, Arisarum vulgare, Cupressus sempervirens, Lepidium sativum, Portulaca oleracea, Allium sativum, Equisetum ramosissimum Zingiber officinale Matricaria aurea, Vitis vinifera Linum pubescens Thymelaea hirsute, Vicia hybrida Lawsonia alba Rubus sanguineus Silybum marianum Mentha longifolia, Polygonum equistiforme Opuntia ficus indica, Corchorus olitorius Artemisia judaica Catha edulis Lavandula officinalis, Achillea fragrantissima Citrus limon Lens esculenta Inula viscosa, Vitex agnus-castus Leontice leontopetalum Ceratonia silique, Desmostachya bipinnata, Calendula officinalis Althaea officinalis Melissa officialil L. Achilloea millefolium Viscum album Teucrium capitatum, Paronichia argentea, Cyperus rotundus Myristica fragrans Olea europaea, Allium cepa Origanum vulgare, Plantago ovata Pistacia palaestina Petroselinum crispum Mentha pulegium, Mentha piperita Cyclamen persicum Rosa phoenicia Ananas comosus, Rumex cyprius, Punica granatum Solanum tuberosum Asparagus aphyllus, Cucurbita Trifolium purpureum Paphanus sativus Urtica plulifera Rosemarinus officinalis, Smilax aspera, Ephedra foemina, Juncus acutus, Chiliadenus iphionodes, Capsella bursa pastoris, Capsella bursa pastoris, Atriplex halimus Thymbra spicata Crataegus aronia Sarcopoterium spinosum, Ecballium elaterium Rhus coriaria Satureja thymbra Laurus nobilis Majorana syriaca, Notobasis syriaca, Quercus ithaburensis, Asphodelus ramosus Tamarindus indica, Camellia sinensis Capparis spinosa Salvia fruticosa Thymus vulgaris, Nicotiana tabacum Lycopersicum esclentum Ammi visnaga, Nicotiana glauca Hypericum triquetrifolium Curcuma longa, Brassica oleracea, Clematis cirrhosa Eminium spiculatum Juglans regia, Haplophyllum tuberculatum Mentha aquatica Nasturtium officinale, Marrubium vulgare, Artemisia sieberi Apium graveolens Matricaria recutita, Ficus carica, Lupinus pilosus, Avena sterilis, Withania somnifera, Malva sylvestris and any combination thereof.

It is another object of the invention to disclose a composition, as defined in any of the above, wherein said cannabis-derived compound is selected from a group consisting from a group consisting of cannabinoids, terpenes, phenolic compounds and any combination thereof.

It is another object of the invention to disclose a composition, as defined in any of the above, wherein cannabinoids are at least one of Tetra-hydro-cannabinoids (d9-THC), Tetra-hydro-cannabinoids (d8-THC), Tetra-hydro-cannabinolic acid (THCA-d9), Tetra-hydro-cannabivarin (THCV/THC-C3), Cannabidiol (CBD), Cannabidiolic acid (CBDA), Cannabidivarin (CBDV), Cannabigerol (CBG), Cannabigerolic acid (CBGA), Cannabinol (CBN), Cannabidiolic acid (CBNA), Cannabichromene (CBC), Cannabichromenic acid (CBCA), and any combination thereof.

It is another object of the invention to disclose a composition, as defined in any of the above, wherein terpenes are mono-terpenes or sesqui-terpenes.

It is another object of the invention to disclose a composition, as defined in any of the above wherein phenolic compounds are at least one of O-glycoside Cannaflavin A, Cannaflavin B, Canabisin D, and any combination thereof.

It is another object of the invention to disclose a composition, as defined in any of the above, wherein said composition is configured to be administrable in a manner selected from a group consisting of an inhaler, a cigarette, a tablet, a capsule, a pill, lyophilized, powder, emulsion, granulated powder, cream, ointment, paste, lotion gel, liquid, a solution, a patch and any combination thereof.

It is another object of the invention to disclose a composition, as defined in any of the above, wherein said composition is configured to be administrable in a manner selected from a group consisting of fast release, slow release, sustained release, controlled release and any combination thereof.

It is another object of the invention to disclose a composition, as defined in any of the above, wherein said composition additionally comprising ingredients selected from a group consisting solubilizers, stabilizers, buffers, tonicity modifiers, bulking agents, viscosity enhancers/reducers, surfactants, chelating agents, adjuvants and any combination thereof.

It is one object of the invention to disclose a method of enhancing a physiological effect of a cannabis-derived compound, comprising the steps of:
a. combining said cannabis-derived compound, at least one portion of at least one plant comprising a methylating/demethylating active agent, and at least one portion of at least one enhancer plant; and
b. administering said synergistic combination to a mammalian subject.

It is another object of the invention to disclose a method, as defined in any of the above, wherein said physiological effect is selected from a group consisting of increased bioavailability, increased appetite, amelioration of a disease condition, and any combination thereof, said disease condition is selected from a group consisting of nausea post chemotherapy, vomiting post chemotherapy, multiple sclerosis, nerve injury, ocular pain, headache, anxiety, insomnia, convulsions, multiple sclerosis, cerebral ischemia Parkinson's disease, epilepsy, osteoarthritis, psoriasis, systemic lupus erythematosus, diabetes, glomerulonephritis, renal ischemia, nephritis, hepatitis, vasculitis, myocardial infarction, cerebral ischemia, inflammatory bowel disease (IBD), colitis, emesis, Crohn's disease, immune-related disorders, pain-related disorders, and any combination thereof.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, is adapted to remain apparent to those skilled in the art, since the generic principles of the present invention have been defined specifically to provide means and methods for enhancing cannabinoids efficacy.

As used herein after, the term “methylation” refers herein after to the addition of a methyl group on a substrate, or the substitution of an atom (or group) by a methyl group. Methylation is a form of alkylation, with a methyl group, rather than a larger carbon chain, replacing a hydrogen atom.

These terms are commonly used in chemistry, biochemistry, soil science, and the biological sciences.

Active DNA demethylation regulates many vital biological processes, including early development and locus-specific gene expression in plants and animals.

In biological systems, methylation is catalyzed by enzymes; such methylation can be involved in modification of heavy metals, regulation of gene expression, regulation of protein function, and RNA processing. In vitro methylation of tissue samples is also one method for reducing certain histological staining artifacts. The counterpart of methylation is called “demethylation”.

In biological systems, methylation is accomplished by enzymes; methylation can modify heavy metals, regulate gene expression, RNA processing and protein function. The Methylation cycle in medicine relates to the metabolism of various systems including the production of glutathione.

As used herein after, the term “cannabis-derived compounds” refers hereinafter to compounds found in cannabis plant, comprising cannabinoids, terpenes and phenolic compounds. As used herein after, the term “cannabinoids” refers hereinafter to a class of diverse chemical compounds which are ligands for cannabinoid receptors in cells that alter neurotransmitter release in the brain. Cannabinoids were primarily discovered in marijuana (cannabis flower) and hashish (compressed cannabis resin) from the plant of Cannabis saliva. This plant contains more than 80 phyto-cannabinoids. The main active constituent of marijuana is the psychoactive Δ9-tetrahydrocannabinol (Δ9-THC), which acts at cannabinoid 1 (CB1) and cannabinoid 2 (CB2) receptors as a partial agonist. Other important natural cannabinoids present in marijuana are the non-psychoactive cannabidiol (CBD), Δ9-tetrahydro-cannabivarin (Δ9-THCV) and cannabichromene (CBC) [1-3]. Among them CBD has attracted the greatest attention thus far. It was shown to antagonize the effects of CB1/CB2 receptor agonists, to counteract the psychotropic and other negative effects of Δ9-THC and several data suggest that it behaves as an inverse agonist of CB1 and CB2 receptors. Some of these plant-derived cannabinoids are used in the medical practice, such as Δ9-THC (dronabinol) and its synthetic analogue, nabilone against chemotherapy-induced nausea and emesis, and as appetite stimulants (e.g. in AIDS patients). CBD combined with Δ9-THC (nabiximols) is used to relief neuropathic pain and spasticity in multiple sclerosis, and as an adjunctive analgesic treatment in advanced cancer pain.

As used herein after, the term “terpenes”, refers hereinafter to a large and diverse class of organic compounds, produced by a variety of plants. Terpenes are derived biosynthetically from units of isoprene, which has the molecular formula C₅H₈. The basic molecular formula of terpenes are multiples of that, (CsHs)n where n is the number of linked isoprene units. Terpenes are fragrant oils that give cannabis its aromatic diversity.

As used herein after, the term Cannabidiol (CBD), refers to Cannabis sativa constituent, which is a pharmacologically broad-spectrum drug that in recent years has drawn increasing interest as a treatment for a range of disorders.

The cannabis derived compounds comprise:
Cannabinoids: Tetra-hydro-cannabinoids (d9-THC), Tetra-hydro-cannabinoids (d8-THC), Tetra-hydro-cannabinolic acid (THCA-d9), Tetra-hydro-cannabivarin (THCV/THC-C3), Cannabidiol (CBD), Cannabidiolic acid (CBDA), Cannabidivarin (CBDV), Cannabigerol (CBG), Cannabigerolic acid (CBGA), Cannabinol (CBN), Cannabidiolic acid (CBNA), Cannabichromene (CBC), Cannabichromenic acid (CBCA);
Terpenes: Mono-terpenes and Sesqui-terpenes; and
Phenolic Compounds: O-glycoside Cannaflavin A Cannaflavin B Canabisin D.

As used herein after, the term “plant portion” refers hereinafter to several structures, found in plants. The plant or plant portions useful in the means and methods of the present invention include but no limited to intact plants, roots, tubers, berries, rhizomes, stems, leaves, flowers, shoots, fruits, grains, or seeds.

The term demethylating agents refers to a group of chemotherapeutic agents with the capacity, both in vitro and in vivo, to induce transient DNA hypomethylation. DNA methylation refers to the addition of a methyl group to a CpG site1. These sites cluster together in areas known as CpG islands and are frequently localized in the proximity of key gene regulatory regions such as gene promoters. DNA methylation, both aberrant and physiologic, of these areas can result in gene silencing and in the equivalent of the physical inactivation, due to either mutations or deletions, of tumor suppressor genes2*. At the present time, two hypomethylating agents are approved in the U.S. and are widely used in Europe and the rest of the world: 5-azacitidine3 and 5-aza-2′-deoxycitidine4 (decitabine). These two agents have significant activity in patients with higher risk myelodysplastic syndromes (MDS). Recently, 5-azacitidine has been reported to improve survival in patients with higher risk MDS in a randomized phase III study.

Plants comprising methylating/demethylating active agents, are used primarily to increase efficacy of administered cannabinoids or cannabis-based compounds and to target the effect of administered cannabinoids to various body parts or physiological systems, such as nervous system, immune modulation, analgesia (such as pain relief, chronic pain, neuropathic pain), arthritis, muscles, skin, respiratory system, cardiovascular system, gastrointestinal tract, body organs, tumors, metastases etc.

Furthermore, the methylating/demethylating agents exert their activities in various body parts, or physiological systems of the treated mamma, thereby targeting their effect to these body parts or physiological systems.

Table 1 discloses the list of plants comprising methylating and demethylating active agents.

TABLE 1
List of methylating and demethylating plants

| English name | Latin Name |
|---|---|
| alfalfa | Medicago sativa |
| Aloe | Aloe succotrina |
| Anabasis | Anabasis alexandri |
| Ancestral | Hordeum Spontaneum |
| Ancestral two-row barley | Hordeum spontaneum |
| Anise seed | Pimpinella Anisum |
| Annual mercury | Mercurialis annua |
| Apple | Malus sylvestris |
| Artichoke | Cynara Scolymus |
| Autumm mandrake | Mandragora autumnalis |
| Bermuda grass | Cynodon Dactylon |
| Bitter gourd | Citrullkus colocynthis |
| Black cumin/nigl | Nigella Sativa |
| Black nightshade | Solaqnum nigrum |
| Blu gum tree | Eucalyptus camaldulensis |
| Cacao | Theobroma Cacao |
| Calliprinos oak | Quercus calliprinos |
| Cape sorrel | Oxalis pes-caprae |
| Caraway Shrubby wormwood | Carum carvi |
| Cardamon | Elettaria cardamomum |
| Carrot | Daucus Carota |
| Chartoc | Sinapis Arvensis |
| Cherry tree | Prunus (cerasus) avium |
| Chives | Allium schoenoprasum |
| Christ thorn jujbe | Ziziphus spina-christi |
| Citron | Citrus medica |
| Clay | Kaolin |
| Clove tree | Eugenia caryophyllata |
| Coffee | Caffea arabica |
| Common fennel | Foeniculum vulgare |
| Common leqorice | Glycyrrhiza glabra |
| Common myrtle | Myrtus communis |
| Common rue | Ruta chalepensis |
| Common sage | Salvia officinalis |
| Common valerian | Valeriana officinalis |
| Coriander | Coriandrum sativum |
| Corn poppy | Papaver umbonatum |
| Corn/maize | Zea mays |
| Crown marigold | Chrysanthemum coronarium |
| Cucumber | Cucumis sativus |
| Cumin seed | Cuminum cyminum |
| Date palm | Phoenix dactylifera |
| Desert origanum | Origanum dayi |
| Dwarf chicory | Cichorium pumilum |
| Eaved madder | Rubia tenuifolia |
| Echinacea | Echinacea purpurea |
| Elder flower | Sambucus nigra |
| European wolfberry | Lycium europaeum |
| Evening primrose | Oenothera drummondii |
| Foxglove | Digitalis purpurea |
| French lavender | Lvandula stoechas |
| Friar's cowl | Arisarum vulgare |
| Funeral cypress | Cupressus sempervirens |
| Garden cress | Lepidium sativum |
| Garden purslane | Portulaca oleracea |
| Garlic | Allium sativum |
| Giant horsetail | Equisetum ramosissimum |
| Ginger | Zingiber officinale |
| Golden chamomile | Matricaria aurea |
| Grapevine | Vitis vinifera |
| Hairy flax | Linum pubescens |
| Hairy thymelaea | Thymelaea hirsuta |
| Hibiscus, sea hibiscus, beach hibiscus, | Hibiscus tiliaceus Linn |
| coastal (or coast) hibiscus, coastal (or | |
| coast) cottonwood, green cottonwood, | |
| nativehibiscus, native rosella, | |
| cottonwood hibiscus | |
| Hairy yellow vetch | Vicia hybrida |
| Hemp | Cannabis sativa |
| Henna plant | Lawsonia alba |
| Holy hramble | Rubus sanguineus |
| Holy thistle | Silybum marianum |
| Horse mint | Mentha longifolia |
| Horseradish | Armoracia rusticana, syn. |
| | Cochlearia armoracia |
| Horsetail knotweed | Polygonum equistiforme |
| Indian fig | Opuntia ficus indica |
| Jeus mallow | Corchorus olitorius |
| Judean wormwood | Artemisia judaica |
| Khat | Catha edulis |
| Lavender | Lavandula officinalis |
| Lavender cotton | Achillea fragrantissima |
| Lemon | Citrus limon |
| Lentil | Lens esculenta |
| Lesser elecampane | Inula viscosa |
| Lilac chastetree | Vitex agnus-castus |
| Lion's Leap | Leontice leontopetalum |
| Locust tree/carob | Ceratonia siliqua |
| Love-grass | Desmostachya bipinnata |
| Marigold | Calendula officinalis |
| Marsh mallow | Althaea officinalis |
| Melissa | Melissa officialil L. |
| Milfoill/Yarrow | Achilloea millefolium |
| Milstletoe | Viscum album |
| Mountain germander | Teucrium capitatum |
| Mountain knotgrass | Paronichia argentea |
| Mild white mustard | Sinapis alba |
| black mustard | Brassica nigra |
| oriental mustard | Brassica juncea |
| Nut grass | Cyperus rotundus |
| Nutmeg tree | Myristica fragrans |
| Olive | Olea europaea |
| Onion | Allium cepa |
| Origano | Origanum vulgare |
| Ovate plantain | Plantago ovata |
| Palestine pistachio | Pistacia palaestina |
| Parsley | Petroselinum crispum |
| Pennyroyal | Mentha pulegium |
| Peppermint | Mentha piperita |
| Persian cyclamen | Cyclamen persicum |
| Phoenician rosa | Rosa phoenicia |
| Pineapple | Ananas comosus |
| Pink sorrel | Rumex cyprius |
| Pomegranat | Punica granatum |
| Potato | Solanum tuberosum |
| Prickly asparagus | Asparagus aphyllus |
| Pumpkin | Cucurbita |
| Purple clover | Trifolium purpureum |
| Radish | Raphanus sativus |
| Black radish; | Raphanus sativus L. var. |
| | niger J. Kern |
| Roman nettle | Urtica plulifera |
| Rosemary | Rosemarinus officinalis |
| Rough hindweed | Smilax aspera |
| Sand cherry | Ephedra foemina |
| Ephedra | Ephedra campilopoda |
| Sharp pointed rush | Juncus acutus |
| Sharp varthemia | Chiliadenus iphionodes |
| Shepherd' purse | Capsella bursa pastoris |
| Shepherd's purse | Capsella bursa pastoris |
| Silvery orache | Atriplex halimus |
| Spiked thymbra | Thymbra spicata |
| Spiny hawthorn | Crataegus aronia |
| Spiny hurnet | Sarcopoterium spinosum |
| Squirting cucumber | Ecballium elaterium |
| Sumach | Rhus coriaria |
| Summer savory | Satureja thymbra |
| Sweet bay | Laurus nobilis |
| Syrian marjoram | Majorana syriaca |
| Syrian thistle | Notobasis syriaca |
| Tabor oak | Quercus ithaburensis |
| Tall asphodel | Asphodelus ramosus |
| Tamarind tree | Tamarindus indica |
| Tea plant | Camellia sinensis |
| Thorny caper | Capparis spinosa |
| Three-lobed sage | Salvia fruticosa |
| Thyme | Thymus vulgaris |
| Tobacco | Nicotiana tabacum |
| Tomato | Lycopersicum esclentum |
| Toothpick | Ammi visnaga |
| Tree tobacco | Nicotiana glauca |
| Tumble st. John'wort | Hypericum triquetrifolium |
| Turmeric | Curcuma longa |
| Turnip/cabbage | Brassica oleracea |
| Virgin's bower | Clematis cirrhosa |
| Wake robin | Eminium spiculatum |
| Walnut | Juglans regia |
| Warty rue | Haplophyllum tuberculatum |
| Water mint | Mentha aquatica |
| Watercress | Nasturtium officinale |
| White horehound | Marrubium vulgare |
| White wormwood | Artemisia sieberi |
| Wild celery | Apium graveolens |
| Wild chamomile | Matricaria recutita |
| Wild fig tree | Ficus carica |
| Wild lupin | Lupinus pilosus |
| Wild oat | Avena sterilis |
| Winter cherry | Withania somnifera |
| Wood mallow | Malva sylvestris |

Enhancers: Enhancer plants are administered along with the methylating and demethylating plants.

Enhancer plants are primarily used to enhance bioavailability. The route of administration is an important determinant of the pharmacokinetics of the cannabinoids in cannabis, particularly absorption and metabolism. Typically, cannabis is smoked as a cigarette. The main advantage of smoking is rapid onset of effect and ease of dose titration. When cannabis is smoked, cannabinoids in the form of an aerosol in the inhaled smoke are absorbed and delivered to the brain rapidly, as would be expected of a highly lipid-soluble drug.

However, smoking is difficult to control, and is not easily standardized. Furthermore, Smoking anything, including cannabis, is not beneficial for the lungs and airway system. A healthier option may be vaporization; because cannabinoids are volatile, they will vaporize at a temperature much lower than actual combustion. Heated air can be drawn through cannabis, the active compounds will vaporize, and these can then be inhaled.

Vaporization delivers the substance in a rapid manner that, like smoking, can be easily titrated to the desired effect. This option removes most of the health hazards of smoking,

Cannabis can also be ingested orally or through a feeding tube. Thought to be safer than smoked cannabis in some ways (e.g., reduced carcinogen exposure), titration of dose through oral administration, unlike smoking, is difficult and may result in stronger and more frequent adverse side effects (e.g., panic, paranoia and performance impairment) This risk stems from slow and unpredictable absorption of orally administered cannabis. Orally ingested THC or cannabis has quite different pharmacokinetics than when it is inhaled. The onset of action is delayed and titration of dosing is more difficult.

Thus, there is a need for absorption enhancers. Non limited examples of plant-derived enhancers is listed in Table 2.

TABLE 2
List of enhancer plants.

| Compound | English name | Latin Name |
|---|---|---|
| BISABOLOL | Chamomile | Maticaria chamimilla |
| A-PINENE | Pine needles | Subgenus Pinus |
| LINALOOL | Lavender | Lavandula officinalis |
| BETA-CARYOPHYLLENE | Black pepper | Piper nigrum |
| MYRCENE | Mango | Mangifera |
| LIMONENE | Lemon tree | Citrus limon |
| LPI | SOY | Glycine max |
| (Lysophosphatidylinositol) | | |
| Palmitoylethanolamide | Fenugreek | Trigonella foenum- |
| | | graecum |
| Resiniferatoxin (RTX) | resin spurge | Euphorbia resinifera, |

As used herein the terms sinusitis or rhinosinusitis, refer hereinafter to inflammation of the paranasal sinuses, the cavities that produce the mucus necessary for the nasal passages to function effectively. Common symptoms include thick nasal mucus, a plugged nose, and facial pain. Other signs and symptoms may include fever, headaches, a poor sense of smell, sore throat, and a cough. Serious complications are rare. Acute sinusitis is defined if it lasts fewer than 4 weeks, and chronic sinusitis—if it lasts for more than 12 weeks. Sinusitis can be caused by infection, allergies, air pollution, or structural problems in the nose. Most cases are caused by a viral infection. A bacterial infection may be present if symptoms last more than 10 days or if a person worsens after starting to improve.

As used herein the term “about” denotes ±25% of the defined amount or measure or value.

### Example 1

The current invention discloses a smoking (or inhaling) composition comprising Cannabidiol (CBD) (or any other cannabinoid) mixed with at least one of herbs/plants of methylating/demethylating plants and at least one of enhancer plants.

Methylating/demethylating plants are used primarily to increase efficacy of administered cannabinoids or cannabis based compounds.

Plants comprising methylating/demethylating active agents are used primarily to increase efficacy of administered cannabinoids or cannabis-based compounds and to target the effect of administered cannabinoids to various body parts or physiological systems, such as nervous system, muscles, skin, gastrointestinal tract immune modulation, pain relief, etc.

The composition is smoked by pre-rolled cigarettes, hand-rolled cigarettes or an herbal mixture for rolling or using an inhaler.

### Example 2

The current example discloses a composition which exerts synergistic effect related to absorption, targeting and physiological effects of cannabinoid or cannabis-related compounds. The relevant effects that may be affected by the current invention includes at least one of the aforementioned physiological effect:

Therapeutic indications for cannabis are assumed to target the endocannabinoid system, which has receptors (CB1 and CB2) involved in cognition, memory, analgesia, psychomotricity, appetite and immune function. There are, however, few data specifically on the efficacy of medicinal cannabinoids for the various indications suggested to date. There are even fewer data for a variety of specific populations such as pregnancy, young people, elderly individuals with complex comorbidities and those with mental health issues. Substantial reliance on research evidence from recreational use of cannabis is therefore required until robust data specifically for medicinal cannabinoids become available. A broad range of therapeutic effects of cannabinoids has been suggested and is currently the focus of much research. Delta-9-tetrahydrocannabinol (THC) has been proposed to be of benefit for chronic pain, nausea and vomiting induced by chemotherapy, and in the reduction of spasms in multiple sclerosis. Cannabidiol (CBD), the second most abundant constituent within cannabis, cannabidiol (CBD), is thought to have a broad range of therapeutic properties, including amelioration of the adverse psychological and cognitive effects of THC. CBD is a low-affinity CB1 and CB2 receptor ligand and negative allosteric modulator of CB1, which reduces the binding of CB1 agonists, while augmenting endocannabinoid tone in an indirect manner. Therapeutic effects of CBD include, but no limited to better cognitive performance, especially memory, and increased gray matter in the hippocampus; neuroprotection, specifically through increased hippocampal cell survival and neurogenesis. Cannabidiol (CBD) has additionally antipsychotic, anxiolytic and anticonvulsant properties and may reduce Parkinsonian symptoms.

The feeling of increased appetite following the use of cannabis has been documented for hundreds of years and is known colloquially as “the munchies” in the English-speaking world. Clinical studies and survey data have found that cannabis increases food enjoyment and interest in food.

In summary, the therapeutic areas currently best associated with exploitation of Cannabis-related medicines include pain, cancer epilepsy, feeding disorders, Parkinson's disease, and immune modulation.

### Example 3

Efficacy in Sinusitis or pain: The current example discloses a composition for amelioration or treatment of sinusitis or pain. The composition comprises a synergistic mixture or formulation of at least one cannabinoid and at least one of the followings plants: black radish (Raphanus sativus L. var. niger J. Kern); mustard (white/yellow mustard, Sinapis alba; brown mustard, Brassica juncea; or black mustard, Brassica nigra); horseradish (Armoracia rusticana, syn. Cochlearia armoracia); Ephedra (Ephedra campilopoda); tea plant (Camellia sinensis).

### Example 4

Efficacy of plant-based formulations in Epilepsy: Epilepsy is a serious brain disorder that could affect anyone at any age and could decrease life expectancy by about 18 years. There are many antiepileptic drugs available worldwide in the market, however around one third of those who develop epilepsy unfortunately continue to experience uncontrolled seizures. Despite the introduction of new antiepileptic drugs, the quality of life and therapeutic response for patients with epilepsy remains still poor. Clinical evidence seems to indicate that CBD is able to manage epilepsy both in adults and children affected by refractory seizures, with a favorable side effect profile. However, to date, clinical trials are both qualitatively and numerically limited, thus yet inconsistent. Therefore, further preclinical and clinical studies are undoubtedly needed to better evaluate the potential therapeutic profile of CBD in epilepsy, although the actually available data is promising.

Objective: The aim of the current study is to assess the effect of a new CBD formulation as seizures protection in the rat model of pilocarpine-induced epilepsy.

Formulation: The plant-based formulation of epilepsy treatment is composed of the following ingredients: (Tables 3A-B)

TABLE 3A
Formulation for epilepsy study

| | Role | Ingredient | Plant source |
|---|---|---|---|
| 1 | Demethylating | Dimethylglycine | Hibiscus |
| | Agent | | tiliaceus Linn |
| 2 | Enhancer (CB1) | Myrcene | Mango |
| | | C10H16 | |
| 3 | Cannabinoid | CBD Cannabidiol | Hemp- |
| | | | Cannabis Sativa |
| 4 | Enhancer (entourage | Palmitoylethanolamide | Fenugreek |
| | compound for | | |
| | endocannabinoids- | | |
| | via CB1 and CB2) | | |

TABLE 3B
Formulation doses for epilepsy study

| | | | | | Dose |
|---|---|---|---|---|---|
| | | Dose Level mg/kg | | | Volume |
| Test Item | Formulation | Low | Mid | High | mL/kg |
| Epilepsy | Dimethylglycine | 125 | 250 | 500 | 5 |
| | CBD | 5.00 | 12.5 | 25.0 | |
| | Myrcene | 0.625 | 1.250 | 2.50 | |
| | Palmitoylethanolamide | 0.625 | 1.250 | 2.50 | |

Study variables and endpoints: The formulation is administered IP (Intraperitoneally). The animals are assessed for:
a. Mortality & morbidity—Twice daily
b. Body weight monitoring—During acclimation and before Test Item (TI) dosing (Day 1)
c. Clinical signs—Before TI dosing, continuous follow-up during the first two hours following Pilocarpine administration. Monitoring of number and duration of seizures during 120 minutes following pilocarpine administration (Seizures rating according to Racine scale).
d. Study termination—At the end of the seizures' observation (120 minutes after Pilocarpine administration) animals are sedated with Ketamine/Xylazine mixture and are subjected to pericardial perfusion with PBS and PFA 4%, brains will be removed and stored in PFA 4% until histopathological analysis

Administration of the Test Items Each Test Item and vehicle formulation are administered to tested animals, according to Table.

TABLE 4
Group Allocation for epilepsy study

| | | Test | Test Item | |
|---|---|---|---|---|
| Treatment | | Item | Dose level | |
| group | Test Item | ROA | (mg/kg) | Pilocarpine |
| 1 | Negative control - | IP | 0 | + |
| | Pilocarpine | | | |
| | only untreated | | | |
| 2 | CBD | | 25 | + |
| 3 | Epilepsy Formulation | | Low | + |
| 4 | Epilepsy Formulation | | Mid | + |
| 5 | Epilepsy Formulation | | High | + |

ROA—route of administration;
NA—not applicable,
IP—intraperitoneal

Administration: Administration of Formulations, Methyl Scopolamine and Pilocarpine is performed as follows:
a. Formulations (“Epilepsy”) are administered IP at three doses (low, mid, high) and the comparator (CBD 25 mg/kg) is administered IP 15 minutes prior to Methyl Scopolamine (same schedule for the vehicle group).
b. Scopolamine methyl-nitrate (1 mg/kg) is injected SC, half an hour before IP administration of pilocarpine (350 mg/kg).
c. Diazepam 10 mg/kg is administered IP in case of status epilepticus (SE)lasting more than 90 minutes.

Clinical Observations: The animals are observed for toxic/adverse symptoms at acclimation and before TI/vehicle/CBD administration and continuously for 120 minutes post pilocarpine administration.

Seizure scoring: Following model induction by Pilocarpine injection, the animals are video recorded for 2 hours. The videos are analyzed and seizures rating are recorded according to the following Racine scale below.
a. Latency to first motor seizure (defined as clonic movement of the fore- and/or hindlimbs for at least 15 seconds duration)
b. Latency to status epilepticus (defined as continuous clonic motor seizure activity for at least 10 min)
c. Number and duration of seizures
d. Mortality rate
e. Seizures rating according to Racine scale (Table 6), given as an accumulated score every 15 min during the 2 hours; at 15, 30, 45, 60, 75, 90, 105, 120 min.
f.

TABLE 5
Racine Scale

| Score | Symptoms |
|---|---|
| 0 | Normal activity |
| 1 | Rigid posture or immobility |
| 2 | Stiffened, extended and often arched tail |
| 3 | Partial body clonus, including forelimb or hind limb clonus or |
| | head bobbing |
| 4 | Whole body continuous clonic seizures with rearing |
| 5 | Severe whole body continuous clonic seizures with rearing and |
| | falling |
| 6 | Status epilepticus. |

Results

The results clearly show significant synergistic efficacy of the Mid dose formulation, which contains only 12.5 mg/kg CBD, in reducing the seizure levels, as compared to vehicle group and to saline group (Student's T-test p-values of 0.02 and 0.05, respectively) and borderline significant efficacy as compared to the CBD (25 mg/kg) group (Student's T-test p value of 0.13).

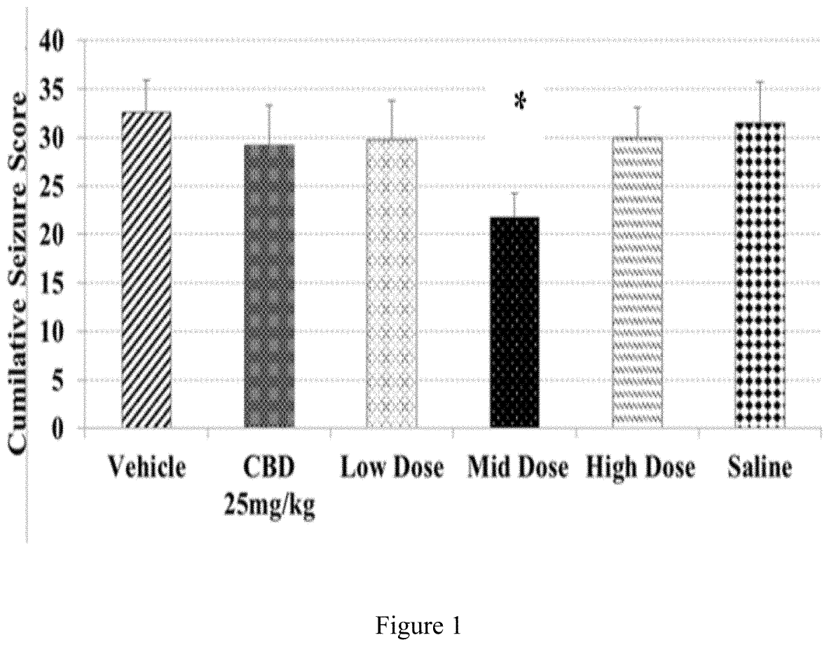

The results are summarized in Table 6 and depicted in FIG. 1. (* Student's T-test, p value<0.05 compared to vehicle-treated animals and compared to saline-treated animals)

TABLE 6
Results of epilepsy study

| | AV- | | | | T-test | T-test | T-test |
|---|---|---|---|---|---|---|---|
| | Seizure | | | | vs. | vs. | vs. |
| | Score | SD | N | SEM | Vehicle | Saline | CBD |
| Vehicle | 32.6 | 9.2 | 8 | 3.3 | | 0.833 | |
| CBD 25 | 29.2 | 10.2 | 6 | 4.2 | 0.52 | 0.70 | |
| mg/kg | | | | | | | |
| Low Dose | 29.8 | 11.3 | 8 | 4.0 | 0.59 | 0.77 | 0.92 |
| Mid Dose | 21.8 | 7.4 | 9 | 2.5 | 0.02 | 0.05 | 0.13 |
| High Dose | 30.0 | 8.8 | 8 | 3.10 | 0.76 | 0.77 | 0.73 |
| Saline | 31.5 | 10.3 | 6 | 4.2 | | | |

### Example 5

Efficacy of plant-based formulations in Lung Cancer: The formulation for studying the efficacy in a lung cancer model is delivered by IP administration

The formulation ingredients are listed in Table 7A-B:

TABLE 7A
Formulation for lung cancer study

| | Role | Ingredient | Plant source |
|---|---|---|---|
| 1 | Demethylating | Hydroxytyrosol | Olive - |
| | Agent | | Olea europaea |
| 2 | Cannabinoid | CBD | Hemp- |
| | | | Cannabis Sativa |
| 3 | Enhancer 1 (CB1) | Myrcene | Mango |
| 4 | Enhancer 2 | Palmitoylethanolamide | Fenugreek |

TABLE 7B
Formulation doses for lung cancer study

| | | | | | Dose |
|---|---|---|---|---|---|
| | | Dose Level mg/kg | | | Volume |
| Test Item | Formulation | Low | Mid | High | 5 mL/kg |
| LC | Hydroxytyrosol | 12.5 | 25.0 | 50.0 | |
| (Lung | CBD | 5.00 | 12.5 | 25.0 | |
| Cancer) | Myrcene | 0.625 | 1.250 | 2.50 | |
| | Palmitoylethanolamide | 0.625 | 1.250 | 2.50 | |

Lung Cancer Model—Study Outline

Female nude mice, are inoculated orthotopically (Lung injection) with lung cancer cells (A549). At tumor detection, mice are randomized to enter the study.

Study Design: The mice are allocated to the treatment groups according to Table 8.

TABLE 8
treatment groups for lung cancer study

| Group | N (Initial) | A549 cells | Treatment |
|---|---|---|---|
| 1 | 10 | + | Vehicle |
| 2 | 10 | + | CBD |
| 3 | 10 | + | Formulation Dose 1 |
| 4 | 10 | + | Formulation Dose 2 |
| 5 | 10 | + | Formulation Dose 3 |
| 6 | 10 | + | Saline |

Treatment: Test items are administered 7 times a week starting Day 4 post inoculation and for at least 14 days.

Animals are examined for
a. Body weight—Three times a week until study termination.
b. Morbidity & mortality check: Daily.
c. Clinical observation—at acclimation, and once a day post inoculation

Termination: is performed at day 14-21 or according to bodyweight loss and morbidity standards.

Followed by necropsy and Lung/Tumor excision, measurement and weight.

### Example 6

Maximum tolerated dose of two plant based formulations (CBD mixtures), following single ip administration to SD rats

The objective of the study was to evaluate the Maximum Tolerated Dosage (MTD) of two CBD mixtures following Intra Peritoneally (IP) administration to Sprague-Dawley (SD) rats.

Study Variables and Endpoints:
a. Mortality & morbidity—Twice a day (once over the weekends).
b. Body weight monitoring—During acclimation, before dosing, twice a week thereafter and before termination (on Day 15).
c. Detailed clinical observations—Before dosing, frequently during the first three hours after dosing, twice a week thereafter and before study termination.
d. Cage side clinical observations—Daily, for three days post administration.
e. Necropsy and Gross pathology—Macroscopic findings on all study animals.
f. Tissue Preservation—Any tissue that displayed pathological changes (in 4% Formaldehyde).

The goal of the current pre-clinical study is to find the Maximal Tolerated Dose (MTD) of two CBD based formulations: Epilepsy formulation and lung cancer (LC) formulation, following a single peritoneal administration to Sprague-Dawley (SD) rats, therapeutic intervention in Epilepsy and Lung Cancer. Dosing is performed in an escalating mode; each dose elevation was dependent on the outcome of the previous dose. According to the results which included body weight and clinical signs assessment, there were no variations in the Tested Items treated rats compared to Vehicle treated rats.

Gross pathology examination based on macroscopic evaluations revealed A-symmetric thymus, in Low-dose-treated rats and slightly enlarged intestine lymph nodes (Peyer's patches), in the Mid-dose-treated rats in both Test Items and Vehicle treated groups.

Since the results were detected in the Vehicle treated rats with no dose relation to the tested items, they were not considered to be related to the Test Items' treatments.

Thus, it can be concluded that a single peritoneal administration of the Test Items to female SD rats, at the highest dose of the 500 mg/kg—for Epilepsy formulation, and 50.0 mg/kg—for LC formulation, is well tolerated and did not cause any significant adverse clinical effects compared to vehicle treatment. This dose may be used as a starting dose for repeated dosing safety evaluation.

Materials and Methods:

Vehicle is composed of Alcohol (20%), Glycerin (5%), PC:PG (5%) Tween 20 (1%) and Distilled water (69%).

Formulation I—‘Epilepsy’ formulation, is composed of Dimethylglycine; CBD; Myrcene and Palmitoylethanolamide, all of which were dissolved in the Vehicle. The dosage of each ingredient in mg/kg is presented in Table 9.

Formulation II—‘Lung Cancer’, is composed of Hydroxytyrosol); CBD; Ephedrin; Myrcene and Palmitoylethanolamide, all of which are dissolved in the Vehicle. The dosage of each ingredient in mg/kg is presented in Table 9.

TABLE 9
The formulations of the MTD study

| | | | | | Dose |
|---|---|---|---|---|---|
| | | Dose Level mg/kg | | | Volume |
| Test Item | Formulation | Low | Mid | High | mL/kg |
| Epilepsy | Dimethylglycine | 125 | 250 | 500 | 5 |
| | CBD | 5.00 | 12.5 | 25.0 | |
| | Myrcene | 0.625 | 1.250 | 2.50 | |
| | Palmitoylethanolamide | 0.625 | 1.250 | 2.50 | |
| Lung | Hydroxytyrosol | 12.5 | 25.0 | 50.0 | |
| Cancer | CBD | 5.00 | 12.5 | 25.0 | |
| (LC) | Myrcene | 0.625 | 1.250 | 2.50 | |
| | Palmitoylethanolamide | 0.625 | 1.250 | 2.50 | |

Results:

Morbidity and Mortality: No morbidity or mortality related to the Test Items was observed in all groups during the in-life period.

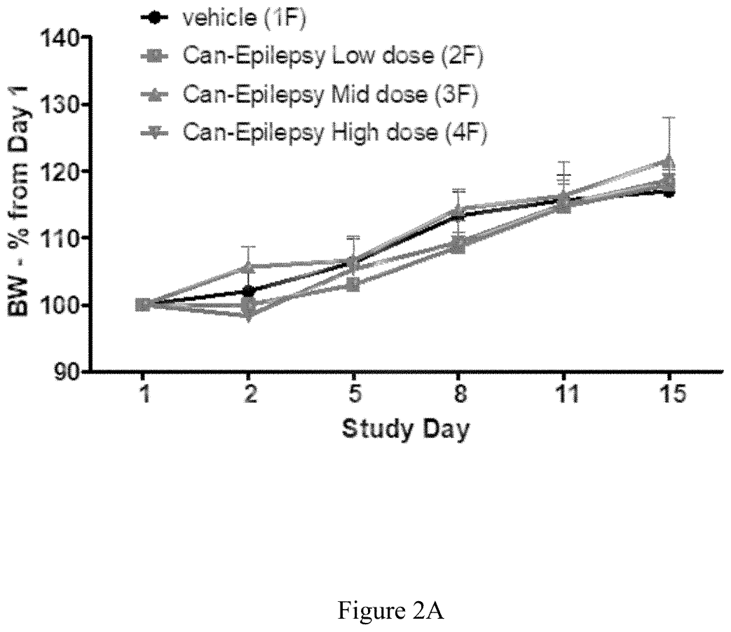

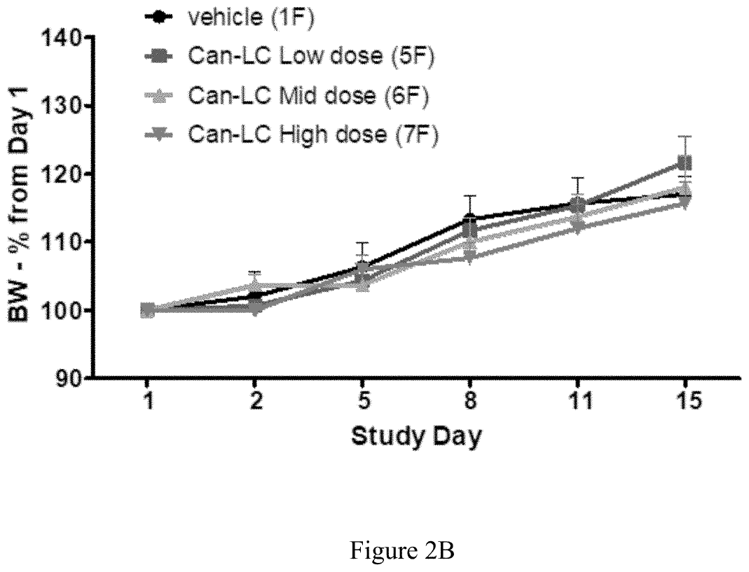

Body weight (BW) was monitored in all animals of all groups. As shown in Figure the average BW gain in the groups treated with both Can-Epilepsy formulation (FIG. 2A) and Can-LC formulation (FIG. 2B), at three doses, low dose groups, mid dose groups and high dose groups is comparable to the Vehicle group. Two-way ANOVA, followed by Bonferroni post-hoc tests reveals no difference between the treatments at all-time points until study termination. FIG. 2 depicts average of BW gain (% of Day 1); FIG. 2A depicts Epilepsy formulation treated rats compared to vehicle. FIG. 2B depicts Lung Cancer formulation treated rats compared to Vehicle.

Detailed and Cage-side Clinical Observations: No clinical sign abnormalities were observed in rats treated with Vehicle group 1F); Can-Epilepsy (Epilepsy formulation) (groups 2F, 3F and 4F) and Can-LC (Lung cancer formulation) (groups 5F, 6F and 7F) during the study. Individual clinical observations are summarized in Table 10.

TABLE 10
Clinical Observations of the MTD study

| | Animal | |
|---|---|---|
| Group | ID | Clinical observation |
| Vehicle (1F) | 1 | NAD on Days 1-15; Euthanized on Day 15 |
| | 2 | NAD on Days 1-15; Euthanized on Day 15 |
| | 3 | NAD on Days 1-15; Euthanized on Day 15 |
| Can-Epilepsy | 4 | NAD on Days 1-15; Euthanized on Day 15 |
| Low dose (2F) | 5 | NAD on Days 1-15; Euthanized on Day 15 |
| | 6 | NAD on Days 1-15; Euthanized on Day 15 |
| Can-Epilepsy | 7 | NAD on Days 1-15; Euthanized on Day 15 |
| Mid dose (3F) | 8 | NAD on Days 1-15; Euthanized on Day 15 |
| | 9 | NAD on Days 1-15; Euthanized on Day 15 |
| Can-Epilepsy | 10 | NAD on Days 1-15; Euthanized on Day 15 |
| High dose (4F) | 11 | NAD on Days 1-15; Euthanized on Day 15 |
| | 12 | NAD on Days 1-15; Euthanized on Day 15 |
| Can-LC | 13 | NAD on Days 1-15; Euthanized on Day 15 |
| Low dose (5F) | 14 | NAD on Days 1-15; Euthanized on Day 15 |
| | 15 | NAD on Days 1-15; Euthanized on Day 15 |
| Can-LC | 16 | NAD on Days 1-15; Euthanized on Day 15 |
| Mid dose (6F) | 17 | NAD on days 1-15; Euthanized on Day 15 |
| | 18 | NAD on days 1-15; Euthanized on Day 15 |
| Can-LC | 19 | NAD on days 1-15; Euthanized on Day 15 |
| High dose (7F) | 20 | NAD on days 1-15; Euthanized on Day 15 |
| | 21 | NAD on days 1-15; Euthanized on Day 15 |

NAD = No Abnormality Detected

Gross Pathology: The rats were evaluated for macroscopic findings in Gross pathology. As shown in Table 5 during the Gross Pathology of Cycle I (Low dose), rats No. 1, 4, and 13 from groups 1F (Vehicle); 2F (Can-Epilepsy) and 5F (Can-LC) had an a-symmetric Thymus, which appeared also in Cycle II in rat No. 2 from the control group (F, Vehicle treated), but not in the treated ones in Cycle II nor in other rats from Cycle III. The tissues were preserved for possible future histology analysis.

Furthermore, in Cycle II (Mid dose), rats No. 8, 9, 16, from groups 3F (Can-Epilepsy) and 6F (Can-LC), had slightly enlarged intestine lymph nodes (Peyer's patches), which appeared also in the control rat No. 2 (group 1F, Vehicle) of Cycle II. These enlarged lymph nodes neither appeared in Cycle I nor III. The tissues were preserved for possible future histology analysis. Results are summarized in Table 11.

TABLE 11
Gross Pathology Evaluation of the MTD study

| | Animal | |
|---|---|---|
| Group | ID | Gross pathology findings |
| Vehicle (1F) | 1 | A-symmetric Thymus, on scheduled |
| | | termination - Day 15 |
| | 2 | A-symmetric Thymus, slightly enlarged |
| | | intestine lymph nodes; on scheduled |
| | | termination - Day 15 |
| | 3 | NAD on scheduled termination - Day 15 |
| Can-Epilepsy | 4 | A-symmetric Thymus, on scheduled |
| Low dose (2F) | | termination - Day 15 |
| | 5 | NAD on scheduled termination - Day 15 |
| | 6 | NAD on scheduled termination - Day 15 |
| Can-Epilepsy | 7 | NAD on scheduled termination - Day 15 |
| Mid dose (3F) | 8 | Slightly enlarged intestine lymph nodes; on |
| | | scheduled termination - Day 15 |
| | 9 | Slightly enlarged intestine lymph nodes; on |
| | | scheduled termination - Day 15 |
| Can-Epilepsy | 10 | NAD on scheduled termination - Day 15 |
| High dose (4F) | 11 | NAD on scheduled termination - Day 15 |
| | 12 | NAD on scheduled termination - Day 15 |
| Can-LC | 13 | A-symmetric Thymus, on scheduled- |
| Low dose (5F) | | termination Day 15 |
| | 14 | NAD on scheduled termination - Day 15 |
| | 15 | NAD on scheduled termination - Day 15 |
| Can-LC | 16 | Slightly enlarged intestine lymph nodes; on |
| Mid dose (6F) | | scheduled termination - Day 15 |
| | 17 | NAD on scheduled termination - Day 15 |
| | 18 | NAD on scheduled termination - Day 15 |
| Can-LC | 19 | NAD on scheduled termination - Day 15 |
| High dose (7F) | 20 | NAD on scheduled termination - Day 15 |
| | 21 | NAD on scheduled termination - Day 15 |

NAD = No Abnormality Detected

Conclusions: In this study a single peritoneal administration of the Test Items, Can-Epilepsy and Can-LC, were evaluated for maximal tolerated dose in female SD rats. BW and clinical signs evaluation demonstrated no variations in the Tested Items treated rats compared to Vehicle treated rats.

Gross pathology examination during termination day revealed A-symmetric thymus in Low-dose-treated rats from both Test Item groups, as well as in the Vehicle treated group. Furthermore, slightly enlarged intestine lymph nodes (Peyer's patches), were observed in the Mid-dose-treated rats from both Test Item groups as well as in the Vehicle treated group. Both findings were detected also in the control—Vehicle treated rats, therefore, they were not considered related to the Test Item treatment.

Based on the above findings and under the conditions of this study, it is concluded that a single peritoneal administration of the Test Items, Can-Epilepsy at dose levels of 125/250/500 mg/kg and Can-LC dose levels of 12.5/25.0/50.0 mg/kg in female SD rats, is well tolerated and does not cause any significant adverse clinical effects compared to Vehicle treatment. For future repeated-dosing safety studies, the highest single dose tested of the Can-Epilepsy and the Can-LC drugs (500 mg/kg and the 50.0 mg/kg respectively) can serve as a starting dose, since they revealed no Test Item related adverse effects under single peritoneal administration

### Example 7

Routes of administration: The combination of a cannabinoid and a methylation agent is formulated to either oral administration, intravenous administration, or topical administration.

The formulations of the present invention comprise inter alia, in a non-limiting matter, additional ingredients or pharmaceutical excipients to further develop a formula to have a desired concentration, effective doses, dosing regiments and treatment times. These ingredients include, inter alia, solubilizers, stabilizers, buffers, tonicity modifiers, bulking agents, viscosity enhancers/reducers, surfactants, chelating agents, and adjuvants.

Cannabis composition of the current invention is administered by smoking a plant-derived cigarette or by oral or intravenous administration. The smoked route is most commonly used is a ready premade-cigarette or hand-rolled cigarette.

Intravenous route offers precise control of dose and timing.

Cannabinoid composition is delivered also by or o-mucosal route.

Nasal administration (through the nose)—can be used for topically acting substances, as well as for insufflation of e.g. decongestant nasal sprays to be taken up along the respiratory tract. Such substances are also called inhalational, e.g. inhalational anesthetics.

Oral administration Oral drugs are taken as tablets or capsules.

Tablets: The dissolution of the tablet can be affected significantly by particle size and crystal form.

The dissolution time can be modified for a rapid effect (fast dissolution) or for sustained release, (slow dissolution rates which prolong the duration of action or avoid initial high plasma levels).

Capsules: A capsule is a gelatinous envelope enclosing the active substance. Capsules can be designed to remain intact for some hours after ingestion in order to delay absorption. They may also contain a mixture of slow- and fast-release particles to produce rapid and sustained absorption in the same dose.

Oral sustained release: Oral sustained release in capsules or tablets is achieved, in a non-limiting matter, by embedding the active ingredient in an insoluble porous matrix, such that the dissolving drug must make its way out of the matrix before it can be absorbed, sustained release formulations in which the matrix swells to form a gel through which the drug exits, or by an osmotic controlled-release oral delivery system, where the active compound is encased in a water-permeable membrane with a laser drilled hole at one end. As water passes through the membrane the drug is pushed out through the hole and into the digestive tract where it can be absorbed.

Solutions: Pharmaceutical solutions are extensively used as dosage forms for the oral administration of therapeutic agents. Pharmaceutical solutions defined as liquid preparations in which the therapeutic agent and the various excipients are dissolved in the chosen solvent system.

Pharmaceutical solutions are homogeneous, i.e. the therapeutic agent(s) and excipients are dissolved in the vehicle Parenteral administration: Parenteral administration is performed using intravenous, subcutaneous, intramuscular, and intra-articular administration. The drug is stored in liquid or if unstable, lyophilized form.

Topical administration: Topical formulations comprise inter alia cream, ointment, paste, lotion or gel.

Transdermal delivery: Transdermal delivery is achieved, for example, by transdermal patches.

Alternative routes of administration are suppository, intraventricular, intramuscular, inhalational, aerosol, and sublingual.

1. A plant-based composition configured to enhance a physiological effect of a cannabis-derived compound, wherein said composition comprises a synergistic combination of said cannabis-derived compound, at least one portion of at least one plant comprising a methylating/demethylating active agent, and at least one portion of at least one enhancer plant.

2. A plant-based composition configured to enhance a physiological effect of a cannabis-derived compound in the treatment of seizures in epilepsy, wherein said composition comprises a synergistic combination of said cannabis-derived compound, at least one portion of at least one plant comprising a methylating/demethylating active agent, and at least one portion of at least one enhancer plant,
wherein said cannabis-derived compound is CBD;
said at least one portion of at least one plant comprising a methylating/demethylating active agent is Hibiscus tiliaceus Linn comprising Dimethylglycine; and
said at least one portion of at least one enhancer plant, is selected from a group consisting of mango, myrcene, palmitoylethanolamide, fenugreek and any combination thereof.

3. (canceled)

4. The composition of claim 1, wherein said physiological effect is selected from a group consisting of increased bioavailability, increased appetite, amelioration of a disease condition, and any combination thereof, said disease condition is selected from a group consisting of nausea post chemotherapy, vomiting post chemotherapy, multiple sclerosis, nerve injury, ocular pain, headache, ADHD, anxiety, insomnia, convulsions, multiple sclerosis, cerebral ischemia, Parkinson's disease, epilepsy osteoarthritis, psoriasis, systemic lupus erythematosus, diabetes, glomerulonephritis, renal ischemia, nephritis, hepatitis, vasculitis, myocardial infarction, inflammatory bowel disease (IBD), colitis, emesis, Crohn's disease, immune-related disorders, pain-related disorders, sinusitis or rhinosinusitis, and any combination thereof.

5. The composition of claim 1, wherein said composition is further configured to target said physiological effect specific body parts or physiological systems.

6. The composition of claim 1, wherein said enhancer plant is selected from a group consisting of Maticaria chamimilla, Subgenus Pinus, Lavandula officinalis, Piper nigrum, Mangifera, Citrus limon, Fenugreek and any combination thereof.

7. The composition of claim 1, wherein said demethylating-methylating plant is selected from a group consisting of: Medicago sativa, Aloe succotrina, Anabasis alexandri, Hordeum Spontaneum, Hordeum spontaneum, Pimpinella Anisum, Mercurialis annua, Malus sylvestris, Cynara Scolymus, Mandragora autumnalis, Cynodon Dactylon, Citrullkus colocynthis, Nigella Sativa, Solaqnum nigrum, Eucalyptus camaldulensis, Theobroma Cacao, Quercus calliprinos, Oxalis pes-caprae, Carum carvi, Elettaria cardamomum, Daucus Carota, Sinapis Arvensis, Prunus (cerasus) avium, Allium schoenoprasum, Ziziphus spina-christi, Citrus medica, Kaolin, Eugenia caryophyllata, Caffea Arabica, Foeniculum vulgare, Glycyrrhiza glabra, Myrtus communis, Ruta chalepensis, Salvia officinalis, Valeriana officinalis, Coriandrum sativum, Papaver umbonatum, Zea mays, Chrysanthemum coronarium, Cucumis sativus, Cuminum cyminum, Phoenix dactylifera, Origanum dayi, Cichorium pumilum Rubia tenuifolia, Echinacea purpurea, Sambucus nigra, Lycium europaeum, Oenothera drummondii, Digitalis purpurea, Lvandula stoechas, Hibiscus, sea hibiscus, beach hibiscus, coastal (or coast) hibiscus, coastal (or coast) cottonwood, green cottonwood, native hibiscus, native rosella, cottonwood hibiscus Hibiscus tiliaceus Linn, Arisarum vulgare, Cupressus sempervirens, Lepidium sativum, Portulaca oleracea, Allium sativum, Equisetum ramosissimum Zingiber officinale Matricaria aurea, Vitis vinifera Linum pubescens Thymelaea hirsute, Vicia hybrida Lawsonia alba Rubus sanguineus Silybum marianum Mentha longifolia, Polygonum equistiforme Opuntia ficus indica, Corchorus olitorius Artemisia judaica Catha edulis Lavandula officinalis, Achillea fragrantissima Citrus limon Lens esculenta Inula viscosa, Vitex agnus-castus Leontice leontopetalum Ceratonia silique, Desmostachya bipinnata, Calendula officinalis Althaea officinalis Melissa officialil L. Achilloea millefolium Viscum album Teucrium capitatum, Paronichia argentea, Cyperus rotundus Myristica fragrans Olea europaea, Allium cepa Origanum vulgare Plantago ovata Pistacia palaestina Petroselinum crispum Mentha pulegium, Mentha piperita Cyclamen persicum Rosa phoenicia Ananas comosus, Rumex cyprius, Punica granatum Solanum tuberosum Asparagus aphyllus, Cucurbita Trifolium purpureum Paphanus sativus Urtica plulifera Rosemarinus officinalis, Smilax aspera, Ephedra foemina, Juncus acutus, Chiliadenus iphionodes, Capsella bursa pastoris, Capsella bursa pastoris, Atriplex halimus Thymbra spicata Crataegus aronia Sarcopoterium spinosum, Ecballium elaterium Rhus coriaria Satureja thymbra Laurus nobilis Majorana syriaca, Notobasis syriaca, Quercus ithaburensis, Asphodelus ramosus Tamarindus indica, Camellia sinensis Capparis spinosa Salvia fruticosa Thymus vulgaris, Nicotiana tabacum Lycopersicum escientum Ammi visnaga, Nicotiana glauca Hypericum triquetrifolium Curcuma longa, Brassica oleracea, Clematis cirrhosa Eminium spiculatum Juglans regia, Haplophyllum tuberculatum Mentha aquatica Nasturtium officinale, Marrubium vulgare, Artemisia sieberi Apium graveolens Matricaria recutita, Ficus carica, Lupinus pilosus, Avena sterilis, Withania somnifera, Malva sylvestris Armoracia rusticana, syn. Cochlearia armoracia, Sinapis alba, Brassica nigra, Brassica juncea, Paphanus Raphanus sativus, Raphanus sativus L. var. niger J. Kern, Ephedra campilopoda and any combination thereof.

8. The composition of claim 1, wherein said cannabis-derived compound is selected from a group consisting from a group consisting of cannabinoids, terpenes, phenolic compounds and any combination thereof.

9. The composition of claim 6, wherein cannabinoids are at least one of Tetra-hydro-cannabinoids (d9-THC), Tetra-hydro-cannabinoids (d8-THC), Tetra-hydro-cannabinolic acid (THCA-d9), Tetra-hydro-cannabivarin (THCV/THC-C3), Cannabidiol (CBD), Cannabidiolic acid (CBDA), Cannabidivarin (CBDV), Cannabigerol (CBG), Cannabigerolic acid (CBGA), Cannabinol (CBN), Cannabidiolic acid (CBNA), Cannabichromene (CBC), Cannabichromenic acid (CBCA), and any combination thereof.

10. The composition of claim 6, wherein terpenes are mono-terpenes or sesqui-terpenes.

11. The composition of claim 6, wherein phenolic compounds are at least one of O-glycoside Cannaflavin A, Cannaflavin B, Canabisin D, and any combination thereof.

12. The composition of claim 1, wherein said composition is configured to be administrable in a manner selected from a group consisting of an inhaler, a cigarette, tablet, a capsule, a pill, lyophilized, powder, emulsion, granulated powder, cream, ointment, paste, lotion gel, liquid, a solution, a patch and any combination thereof.

13. The composition of claim 1, wherein said composition is configured to be administrable in a manner selected from a group consisting of fast release, slow release, sustained release, controlled release and any combination thereof.

14. The composition of claim 1, wherein said composition additionally comprising ingredients selected from a group consisting solubilizers, stabilizers, buffers, tonicity modifiers, bulking agents, viscosity enhancers/reducers, surfactants, chelating agents, adjuvants and any combination thereof.

15. A method of enhancing a physiological effect of a cannabis-derived compound, comprising the steps of:
a. combining said cannabis-derived compound, at least one portion of at least one plant comprising a methylating/demethylating active agent, and at least one portion of at least one enhancer plant; and,
b. administering said synergistic combination to a mammalian subject.

16. The method of claim 15, wherein said physiological effect is selected from a group consisting of increased bioavailability, increased appetite, amelioration of a disease condition, and any combination thereof, said disease condition is selected from a group consisting of nausea post chemotherapy, vomiting post chemotherapy, multiple sclerosis, nerve injury, ocular pain, headache, anxiety, insomnia, convulsions, multiple sclerosis, cerebral ischemia Parkinson's disease, epilepsy, osteoarthritis, psoriasis, systemic lupus erythematosus, diabetes, glomerulonephritis, renal ischemia, nephritis, hepatitis, vasculitis, myocardial infarction, cerebral ischemia, inflammatory bowel disease (IBD), colitis, emesis, Crohn's disease, immune-related disorders, pain-related disorders, sinusitis or rhinosinusitis, and any combination thereof.

17. The composition of claim 1, wherein said cannabis-derived compound is CBD;
said at least one portion of at least one plant comprising a methylating/demethylating active agent is olive, comprising Hydroxytyrosol; and
said at least one portion of at least one enhancer plant, is selected from a group consisting of mango, myrcene, palmitoylethanolamide, fenugreek and any combination thereof, for use in the treatment of lung cancer.
